(19) United States
(12) Patent Application Publication
(10) Pub. No.: US 2014/0336523 A1
(43) Pub. Date: Nov. 13, 2014

(54) ESTIMATION OF ENERGY EXPENDITURE

(71) Applicant: TECOM AS, Bergen (NO)

(72) Inventors: Ole Brix, Isdalsto (NO); Hans Flaatten, Bergen (NO); Anne Berit Guttormsen, Bergen (NO)

(73) Assignee: TECOM AS, Bergen (NO)

(21) Appl. No.: 14/363,159

(22) PCT Filed: Dec. 5, 2012
(86) PCT No.: PCT/EP2012/074523

§ 371 Date: Jun. 5, 2014

(30) Foreign Application Priority Data
Dec. 6, 2011 (GB) 1120909.5

(51) Int. Cl. A61B 5/083; A61B 5/091; A61B 5/00

(52) U.S. Cl. CPC A61B 5/0833; A61B 5/0836; A61B 5/6887; A61B 5/091
USPC 600/531

(57) ABSTRACT

An apparatus (1) for estimating the energy expenditure of a patient (2) comprises means (3) for receiving a set of measurements (6) from a ventilator (4), wherein the set of measurements (6) comprises at least one gas concentration measurement. The apparatus (1) further comprises means (7) for estimating the energy expenditure of the patient (2) based on the set of measurements (6).

### FIELD OF THE INVENTION

The present invention relates to estimating the energy expenditure of a patient. In particular, the invention relates to estimating the energy expenditure of a patient receiving assisted respiration from a ventilator.

### BACKGROUND OF THE INVENTION

A mechanical ventilator is an apparatus which provides breathing assistance to a patient who is physically unable to breathe sufficiently. Mechanical ventilators are often referred to as respirators, or simply as ventilators. The ventilator provides breathing assistance by mechanically moving air into and out of the patient's lungs. Modern ventilators tend to be computerised devices and are used in intensive care medicine, home care, emergency medicine and anaesthesia. Such a ventilator typically comprises a gas reservoir or turbine and air and oxygen supplies. The respiratory gases are transported between the ventilator and the patient via disposable or reusable pulmonary tubes. The pulmonary tubes are a conduit which is in fluid communication with both the ventilator and the patient in order to transport the inspired and expired gases therebetween. To be in fluid communication with the patient, the pulmonary tube commonly comprises either a face mask or a tracheal tube. The ventilator may additionally include a humidifier, water traps, a nebulizer, sensors, and various connectors and valves. By means of the sensors, the ventilator is able to monitor certain patient-related parameters (such as pressure, volume and flow) in order to ensure that the patient is receiving the correct respiration assistance for his or her physiology.

In an intensive care unit (ICU), information regarding a patient's metabolism is important for determining the correct amount of clinical nutrition needed. This is especially important for ICU patients, who are generally unable to ingest food, and whose nutritional requirements must be met with the highest possible accuracy in order to avoid underfeeding or overfeeding. The energy expenditure of a patient can be estimated using existing empirical models, but these estimates may not be accurate in the case of a mechanically ventilated patient, whose condition tends to change drastically over time.

An indirect calorimeter may be employed to assess a patient's energy expenditure. An indirect calorimeter measures various properties of the air inhaled and exhaled by the patient in order to estimate the energy expended by the patient's metabolism. However, it is extremely dangerous to perform such measurements on patients who are receiving respiratory assistance from an ventilator since they are often in a critical state of health. Disconnecting such a patient from the ventilator, even for a short amount of time, to conduct measurements with an indirect calorimeter would put the patient's life at great risk and is highly undesirable.

U.S. Pat. No. 5,072,737 describes a method and apparatus for measuring metabolic rates of a patient intubated on a ventilator. An inspiration sample of gases provided by the ventilator is collected. End-tidal and ambient pressure samples of gases exhaled by the patient are also collected. A sensor means is provided external to the ventilator and is arranged to receive gas samples via three different conduits. The sensor means includes an oxygen sensor, a carbon dioxide sensor and a pressure sensor, and provides a signal indicative of an unknown parameter of a gas sample. A computer uses information from the various sensors to compute breath-by-breath flow weighted average rates of oxygen consumption and carbon dioxide elimination. In other words, according to U.S. Pat. No. 5,072,737, the ventilator is solely used to measure the flow rate of exhaled gas, whilst all the other measurements are taken by means of external sensors. Thus, the apparatus of U.S. Pat. No. 5,072,737 requires numerous additional conduits and sensors to be connected to the ventilator.

### SUMMARY OF THE INVENTION

It is a preferred aim of the invention to overcome or mitigate the problems and disadvantages described above.

A first aspect of the invention provides an apparatus for estimating energy expenditure of a patient, the apparatus comprising: means for receiving a set of measurements from a ventilator, wherein the set of measurements comprises at least one gas concentration measurement; and means for estimating the energy expenditure of the patient based on the set of measurements. The term “energy expenditure” used herein is preferably understood to refer to the metabolic energy expenditure of the patient.

Hence, the apparatus advantageously uses measurements that are already available from a ventilator to estimate the energy expenditure of a patient. This avoids the need for a separate indirect calorimeter, which would require periodic and laborious calibration. Furthermore, the apparatus is also simple to use for staff in an intensive care unit, because it makes use of a ventilator with which they are already familiar. Yet further, the apparatus avoids the need to insert an external device into the pulmonary circuit between the ventilator and the patient, and thereby reduces the possibility for inaccuracy caused by perturbation of thermodynamic and mechanical parameters that may result from inserting an external device into the pulmonary circuit.

The at least one gas concentration measurement preferably comprises an expiratory carbon dioxide concentration measurement and/or an inspiratory oxygen concentration measurement. The apparatus preferably further comprises means for estimating an inspiratory carbon dioxide concentration based on the inspiratory oxygen concentration measurement. The apparatus preferably further comprises means for estimating an inspiratory carbon dioxide concentration based on a known concentration of carbon dioxide in medical air. The means for estimating the energy expenditure is preferably operable to estimate the energy expenditure based on an estimate of inspiratory carbon dioxide concentration. Estimating the inspiratory carbon dioxide concentration avoids the need for a sensor to measure inspiratory carbon dioxide concentration, thereby simplifying the apparatus. Estimating the inspiratory carbon dioxide concentration also avoids the difficulties associated with measuring the inspiratory carbon dioxide concentration, which is too small to measure reliably.

The apparatus preferably further comprises means for receiving an expiratory oxygen concentration measurement, wherein the means for estimating the energy expenditure is operable to estimate the energy expenditure based on the set of measurements and the expiratory oxygen concentration measurement. The apparatus is operable to receive the expiratory oxygen concentration measurement from a sensor that is separate from the ventilator. In this context, the term “separate from the ventilator” is preferably understood to mean that the sensor is not part of the ventilator. To put this another way, the sensor is preferably an additional component that is not supplied with the ventilator, but which is added to allow the energy expenditure of the patient to be estimated. By making use of measurements that are already available from a ventilator, the apparatus advantageously requires only one additional sensor to measure expiratory oxygen concentration. This simplifies the construction and reduces the cost of the apparatus. Preferably, the sensor for measuring oxygen concentration is located in a pulmonary tube, in close proximity to the patient. The close proximity of the sensor to the patient improves the accuracy of the expiratory oxygen concentration measurement. Alternatively, the apparatus could be operable to receive the expiratory oxygen concentration measurement from the ventilator itself; in this case, the set of measurements from the ventilator would further comprise an expiratory oxygen concentration measurement.

The set of measurements preferably further comprises an expiratory volume measurement and/or an inspiratory volume measurement. The set of measurements more preferably comprises only one of an expiratory volume measurement and an inspiratory volume measurement. The apparatus preferably further comprises means for estimating an inspiratory volume based on the set of measurements. More specifically, the apparatus can estimate the inspiratory volume based on an expiratory volume measurement, an inspiratory oxygen concentration measurement, an expiratory carbon dioxide concentration measurement and an expiratory oxygen concentration measurement. Alternatively, the apparatus preferably further comprises means for estimating an expiratory volume based on the set of measurements. More specifically, the apparatus can estimate the expiratory volume based on an inspiratory volume measurement, an inspiratory oxygen concentration measurement, an expiratory carbon dioxide concentration measurement and an expiratory oxygen concentration measurement. Estimating the inspiratory volume or expiratory volume improves the accuracy of the estimate of the patient's energy expenditure. Estimating one of the inspiratory volume or expiratory volume can also avoid the need for a sensor to measure the other volume, thereby simplifying the apparatus. Preferably, the estimate of inspiratory carbon dioxide concentration is also used to estimate the inspiratory volume or the expiratory volume. The means for estimating the energy expenditure is preferably further operable to estimate the energy expenditure of the patient based on an estimate of inspiratory volume or an estimate of expiratory volume.

The apparatus preferably further comprises means for correcting a measurement in the set of measurements to produce a corrected measurement, wherein the corrected measurement compensates for a difference between a thermodynamic condition at the ventilator and a respective thermodynamic condition at the patient, and wherein the means for estimating the energy expenditure of the patient is further operable to estimate the energy expenditure based on the corrected measurement. By correcting a measurement in this manner, the accuracy of the estimated energy expenditure can be improved. The corrected measurement preferably compensates for a difference in temperature and/or relative humidity. The means for correcting a measurement is preferably operable to correct an expiratory volume measurement or an inspiratory volume measurement.

The set of measurements preferably further comprises a respiratory frequency. Respiratory frequency is another measurement that is already available from many existing ventilators, and which can advantageously be used to estimate the daily energy expenditure of the patient.

The apparatus can be integrated with the ventilator. Alternatively, the apparatus can be detachably connectable to the ventilator. In the latter case, the apparatus can be retrofitted to an existing ventilator, or supplied as an optional add-on unit, to provide the additional functionality of measuring the energy expenditure of a patient.

A further aspect of the invention provides a method for estimating energy expenditure of a patient, the method comprising: receiving a set of measurements from a ventilator, wherein the set of measurements comprises at least one gas concentration measurement; and estimating the energy expenditure of the patient based on the set of measurements. The at least one gas concentration measurement preferably comprises an expiratory carbon dioxide concentration measurement and/or an inspiratory oxygen concentration measurement.

The method preferably further comprises estimating an inspiratory carbon dioxide concentration based on the inspiratory oxygen concentration measurement. The method preferably further comprises estimating an inspiratory carbon dioxide concentration based on a known concentration of carbon dioxide in medical air. The step of estimating the energy expenditure is preferably based on an estimate of inspiratory carbon dioxide concentration.

The method preferably further comprises receiving an expiratory oxygen concentration measurement, and the step of estimating the energy expenditure preferably comprises estimating the energy expenditure based on the set of measurements and the expiratory oxygen concentration measurement. The expiratory oxygen concentration measurement is preferably received from a sensor that is separate from the ventilator.

The set of measurements preferably further comprises an expiratory volume measurement or an inspiratory volume measurement. The method preferably further comprises estimating an inspiratory volume or an expiratory volume based on the set of measurements. The step of estimating the energy expenditure is preferably based on an estimate of inspiratory volume or an estimate of expiratory volume.

The method preferably further comprises: correcting a measurement in the set of measurements to produce a corrected measurement, wherein the corrected measurement compensates for a difference between a thermodynamic condition at the ventilator and a respective thermodynamic condition at the patient, and wherein the step of estimating the energy expenditure of the patient further comprises estimating the energy expenditure based on the corrected measurement. The corrected measurement preferably compensates for a difference in temperature and/or relative humidity. The set of measurements preferably further comprises an expiratory volume measurement, and the step of correcting a measurement preferably comprises correcting the expiratory volume measurement. The set of measurements preferably further comprises a respiratory frequency. The method can be performed by the ventilator itself, or by an apparatus that is detachably connected to the ventilator.

A further aspect of the invention provides a processor-readable medium comprising instructions which, when executed by a processor, cause the processor to perform a method for estimating energy expenditure of a patient, the method comprising: receiving a set of measurements from a ventilator, wherein the set of measurements comprises at least one gas concentration measurement; and estimating the energy expenditure of the patient based on the set of measurements. The processor-readable medium can be integrated with the ventilator.

A further aspect of the invention provides an apparatus substantially as described herein and/or as illustrated in any of the accompanying drawings. A further aspect of the invention provides a method substantially as described herein and/or as illustrated in any of the accompanying drawings.

### BRIEF DESCRIPTION OF THE DRAWINGS

Preferred features of the invention will now be described, purely by way of example, with reference to the accompanying drawings, wherein like elements are indicated using like reference signs, and in which:

FIG. 1 is a schematic diagram illustrating an apparatus for estimating the energy expenditure of a patient in accordance with the present invention;

FIG. 2 is a schematic diagram illustrating thermodynamic conditions at a patient and a ventilator;

FIG. 3 is an example of a user interface for the apparatus shown in FIG. 1;

FIG. 4 is a flow chart of a method in accordance with the present invention; and

FIG. 5 is a schematic diagram of a computer system that may be used to implement a method in accordance with the present invention.

### DETAILED DESCRIPTION

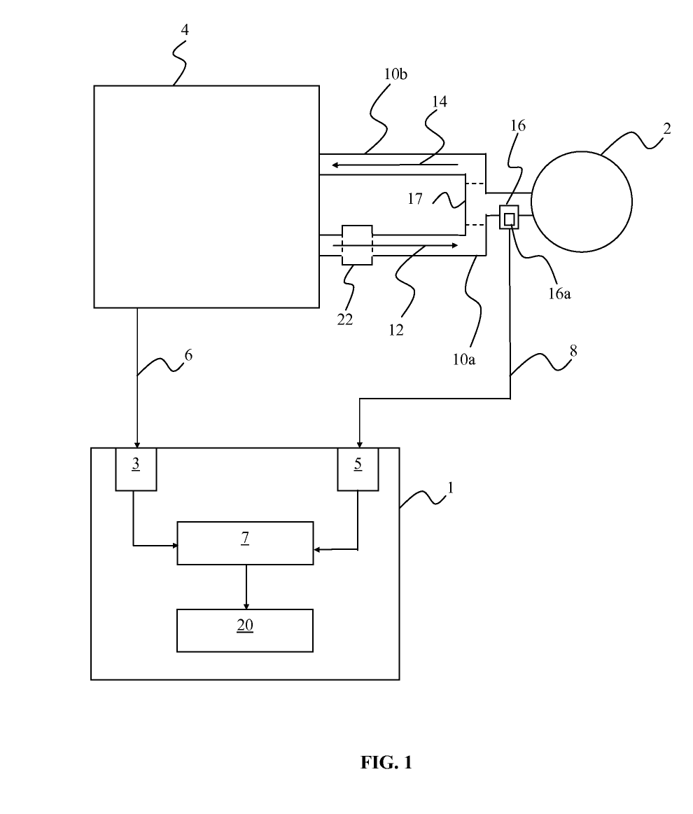

FIG. 1 illustrates an apparatus 1 for estimating the energy expenditure of a patient 2. The apparatus 1 is coupled to a ventilator 4. In use, the ventilator 4 is coupled to the patient 2 by pulmonary tubes 10, thereby to allow the patient 2 to receive an inspiratory gas 12 provided by the ventilator 4 and to allow the return of an expiratory gas 14 to the ventilator 4. The pulmonary tubes 10 comprise a first pulmonary tube 10a to conduct the inspiratory gas 12 and a second pulmonary tube 10b to conduct the expiratory gas 14. The first and second pulmonary tubes 10a, 10b are connected by a joint 17 near the patient 2. The joint 17 may comprise a T-piece or a Y-piece. A humidifier 22 may optionally be connected to the first pulmonary tube 10a to increase the humidity of the inspiratory gas 12.

The apparatus 1 includes a first input 3, a second input 5, a processing means 7 and a display 20. The first input 3 is arranged to receive a set of measurements 6 from the ventilator 4. The second input 5 is arranged to receive a further set of measurements 8 from one or more sensors 16 that are separate from the ventilator 4. The first input 3 and the second input 5 are coupled to a processing means 7. The processing means 7 is operable to perform calculations, as described in more detail below, including calculations to estimate the energy expenditure of the patient 2 based on the set of measurements 6. The processing means 7 could comprise a personal computer, a microprocessor, a microcontroller, a digital signal processor, programmable logic, software, firmware and/or any other means suitable for estimating the energy expenditure of the patient.

The display 20 is operable to present various measurements and estimates to a user. In particular, the display 20 is operable to display the energy expenditure estimated by the processing means 7. The display 20 may also be operable to display any measurement selected from the set of measurements 6 from the ventilator 4 and/or the further set of measurements 8 from the one or more sensors 16. The display 20 may be the monitor of a personal computer used to implement the processing means 7.

In the example shown in FIG. 1, the apparatus 1 is illustrated as being a separate entity from the ventilator 4. In such an example, the apparatus 1 is detachably coupled to the ventilator 4. For example, the apparatus 1 may be implemented using a personal computer, which can be coupled to the ventilator 4 by a suitable communication interface. The communication interface may be a wired communication interface (such as an Ethernet, serial port or universal serial bus (USB) interface) or a wireless communication interface (such as an IEEE 802.11 (Wi-Fi®) or Bluetooth® interface).

In other examples, the apparatus 1 can be integrated with the ventilator 4. In these examples, the processing means 7 may comprise the processor that is usually used by the ventilator 4 to monitor and control the patient's breathing, whilst the display 20 may comprise the display that is usually used by the ventilator 4 to display data relating to the patient's breathing.

In use, the ventilator 4 measures various properties of the inspiratory gas 12 and the expiratory gas 14 in order to ensure that the patient is receiving appropriate breathing assistance. Of the many measurements that are made by the ventilator 4, the following measurements are of particular relevance to the present invention:
expiratory volume (Vₑ), which is a measurement of the total volume of air exhaled by the patient (typically measured in millilitres);
inspiratory volume (Vᵢ) which is a measurement of the total volume of air inhaled by the patient (typically measured in millilitres);
inspiratory oxygen concentration (Fᵢ_{_{O2}}), which is a measurement of the proportion of oxygen in the air inhaled by the patient (expressed as a percentage);
expiratory carbon dioxide concentration (Fₑ_{_{CO2}}), which is a measurement of the proportion of carbon dioxide in the air exhaled by the patient (expressed as a percentage); and
breathing frequency (f), which is a measurement of the number of breaths taken by the patient per unit time (typically measured in breaths per minute).

These measurements are made by the ventilator 4 and provided to the first input 3 of the apparatus 1 as the set of measurements 6. An example of a ventilator 4 that is suitable for providing the set of measurements 6 is the Dräger Evita® XL, manufactured by Drägerwerk AG & Co. Other suitable ventilators 4 could be used. In an example in which the ventilator 4 is a Dräger Evita® XL (or another ventilator with similar functionality), the apparatus 1 can be coupled to the ventilator 4 by an RS-232 connection, and the ventilator 4 can send the set of measurements 6 to the apparatus 1 using the LUST protocol. The LUST protocol is a proprietary protocol that is implemented in the Dräger Evita® XL, and is capable of sending four types of information: identification information; status information; data; and alarms. In this example, measurements of Vₑ, Fᵢ_{_{O2}} and Fₑ_{_{CO2}} are included in the identification information that is communicated from the ventilator 4 to the apparatus 1. A measurement of the pressure at the end of the patient's exhalation can also be included in the identification information that is communicated from the ventilator 4 to the apparatus 1.

Whilst known ventilators can measure many different properties of the inspiratory gas 12 and the expiratory gas 14, they are not designed to measure the expiratory oxygen concentration or the inspiratory carbon dioxide concentration because these measurements are not considered to be useful for achieving the ventilator's primary purpose of ensuring that the patient receives appropriate breathing assistance. The expiratory oxygen concentration (Fₑ_{_{O2}}) is a measurement of the proportion of oxygen in the air exhaled by the patient (expressed as a percentage). The inspiratory carbon dioxide concentration (Fᵢ_{_{CO2}}) is a measurement of the proportion of carbon dioxide in the air inhaled by the patient (expressed as a percentage).

Since known ventilators do not measure the expiratory oxygen concentration, the sensors 16 preferably comprise an oxygen concentration sensor 16a for measuring the expiratory oxygen concentration. Suitable oxygen concentration sensors are known in the art. Purely by way of example, the oxygen concentration sensor 16a may be an AX300-I Portable Oxygen Analyzer, manufactured by Teledyne Analytical Instruments of California, USA. The second input 5 is arranged to receive an expiratory oxygen concentration measurement from the oxygen concentration sensor 16a. In the example shown in FIG. 1, the oxygen concentration sensor 16a is separate from the ventilator 4.

The oxygen concentration sensor 16a is preferably located within the joint 17. Locating the oxygen concentration sensor 16a within the joint 17 allows the expiratory oxygen concentration to be measured very close to the patient 2, which means that the expiratory oxygen concentration is measured under substantially the same thermodynamic conditions that exist at the patient. This avoids the need to correct the expiratory oxygen concentration measurement to compensate for differences in thermodynamic conditions between the patient and the point at which it is measured. For similar reasons, if the sensors 16 comprise any sensors other than the oxygen concentration sensor 16a, these are also preferably located within the joint 17.

Although ventilators that are currently on the market are not capable of measuring expiratory oxygen concentration, future ventilators may be arranged to measure expiratory oxygen concentration. For example, future ventilators may comprise an oxygen concentration sensor for the specific purpose of measuring expiratory oxygen concentration, or they may use an existing sensor for measuring inspiratory oxygen concentration for the further purpose of measuring expiratory oxygen concentration. The first input 3 of the apparatus 1 would then receive the expiratory oxygen concentration measurement from the ventilator 4. The present invention preferably encompasses apparatuses and methods for estimating energy expenditure based on an expiratory oxygen concentration measurement that is received from the ventilator 4.

The apparatus 1 could also be used with less-sophisticated ventilators that are not necessarily able to measure each of the expiratory volume, inspiratory oxygen concentration, expiratory carbon dioxide concentration and breathing frequency. When used with such less-sophisticated ventilators, the one or more sensors 16 will comprise one or more additional sensors to measure the property that is not measured by the ventilator. However, at the very least, it is envisaged that the apparatus 1 will be used with a ventilator 4 that is capable of providing at least one gas concentration measurement; the at least one gas concentration measurement could be any one or more of an inspiratory oxygen concentration, an expiratory carbon dioxide concentration or even an expiratory oxygen concentration, depending on the capabilities of the ventilator 4.

For the sake of clarity, the following description will assume that the ventilator 4 is capable of measuring the expiratory volume, the inspiratory oxygen concentration, expiratory carbon dioxide concentration and breathing frequency, and that the expiratory oxygen concentration measurement is received from a sensor 16a that is separate from the ventilator 4, although it will now be apparent that the invention is preferably not limited to this particular arrangement.

The processing means 7 is operable to estimate the energy expenditure of the patient 2 in the following manner. In order to estimate the energy expenditure, it is necessary to know the oxygen elimination (\dot{V}_{O}_{₂}) and carbon dioxide production (\dot{V}_{CO}_{₂}), which may be expressed by the following equations:

\dot{V}_{CO}_{₂}=Vₑ×Fₑ_{_{CO2}}−Vᵢ×Fᵢ_{_{CO2}} (1)

\dot{V}_{O}_{₂}=Vᵢ×Fᵢ_{_{O2}}−Vₑ×Fₑ_{_{O2}} (2)

As explained above, Vₑ is the expiratory volume, Vᵢ is the inspiratory volume, Fᵢ_{_{CO2}} is the inspiratory carbon dioxide concentration, Fₑ_{_{CO2}} is the expiratory carbon dioxide concentration, Fᵢ_{_{O2}} is the inspiratory oxygen concentration and Fₑ_{_{O2}} is the expiratory oxygen concentration.

Using the values for \dot{V}_{CO}_{₂} and \dot{V}_{O}_{₂} calculated in accordance with equations (1) and (2) respectively, the energy expenditure of the patient 2 is then calculated using the Weir formula (Weir, 1949):

EE=3.9×\dot{V}_{O}_{₂}+1.1×\dot{V}_{CO}_{₂} (3)

where EE is the energy expenditure measured in kilocalories per breath.

It can be seen from equations (1), (2) and (3) that calculation of the patient's energy expenditure involves six variables, i.e. Vᵢ, Vₑ, Fᵢ_{_{CO2}}, Fₑ_{_{CO2}}, Fᵢ_{_{O2}} and Fₑ_{_{O2}}. The inventors have discovered that the patient's energy expenditure can be estimated reliably using measurements of Vₑ, Fₑ_{_{CO2}} and Fᵢ_{_{O2}} made by the ventilator 4, plus a measurement of Fₑ_{_{O2}} provided by the oxygen concentration sensor 16a. Thus, the need for a separate indirect calorimeter can be avoided, and the number of sensors can be reduced, by making use of measurements that are already available from the ventilator 4.

As mentioned above, known ventilators do not measure the inspiratory carbon dioxide concentration, Fᵢ_{_{CO2}}. Furthermore, the inspiratory carbon dioxide concentration is so small that it is difficult to measure reliably. The inventors have discovered that the patient's energy expenditure can be estimated reliably, without measuring the inspiratory carbon dioxide concentration, based upon an estimate of the inspiratory carbon dioxide concentration. By way of explanation, the inspiratory gas 12 usually comprises medical air mixed with oxygen in a known ratio; that is, in addition to the oxygen that is already present in the medical air, the inspiratory gas 12 comprises a known amount of supplementary oxygen. The concentration of carbon dioxide in medical air is known a priori. For example, the medical dry air that is commonly found in hospitals and provided to the patient 2 by the ventilator 4 typically has a carbon dioxide concentration of 0.039%. Thus, it is possible to calculate the inspiratory carbon dioxide concentration based upon an inspiratory oxygen concentration measurement, the known ratio between medical air and supplementary oxygen in the inspiratory gas 12, and the known concentration of carbon dioxide in medial air. The processing means 7 is preferably operable to calculate the inspiratory carbon dioxide concentration as a function of the inspiratory oxygen concentration that is measured by the ventilator 4. For example, when the carbon dioxide concentration is assumed to be 0.039%, the inspiratory carbon dioxide concentration can be calculated using the following equation:

Fᵢ_{CO2}=0.039×(120.95−Fᵢ_{_{O2}}) (4)

The resulting estimate of the inspiratory carbon dioxide concentration can be used as the value for Fᵢ_{CO2} in equation (1). This advantageously avoids the need for a sensor to measure the inspiratory carbon dioxide concentration. Furthermore, this also avoids the error in the estimate of the patient's energy expenditure that would result from the inherent inaccuracy of measuring the inspiratory carbon dioxide concentration directly.

Whilst some ventilators (such as the Dräger Evita® XL) are capable of measuring the inspiratory volume, Vᵢ, it is preferable not to use a measurement of the inspiratory volume to estimate the patient's energy expenditure. This is because the presence of errors in the measurements of both expiratory volume and inspiratory volume would result in a large error in the estimated energy expenditure. The inventors have discovered that the patient's energy expenditure can be estimated more accurately, without measuring the inspiratory volume, based upon an estimate of the inspiratory volume. Hence, the processing means 7 is preferably operable to calculate the inspiratory volume as a function of the measured expiratory volume, the measured expiratory oxygen concentration, the measured expiratory carbon dioxide concentration, the measured inspiratory oxygen concentration and the estimated inspiratory carbon dioxide concentration. The inspiratory volume can be calculated using the Haldane transformation:

$${V}_{i} = \frac{{V}_{e} ( 1 - {F}_{{e}_{O 2}} - {F}_{{e}_{CO 2}} )}{1 - {F}_{{i}_{O 2}} - {F}_{{i}_{CO 2}}} \qquad ( 5 )$$

Calculating the inspiratory volume as a function of the measured expiratory volume in this manner has the effect of correlating the error in the inspiratory and expiratory volumes, which reduces the error in the estimate of the energy expenditure of the patient.

Alternatively, the patient's energy expenditure can be estimated based upon an estimate of the expiratory volume. In this case, the inspiratory volume is measured by the ventilator 4, and the processing means 7 is operable to calculate the expiratory volume as a function of the measured inspiratory volume, the measured expiratory oxygen concentration, the measured expiratory carbon dioxide concentration, the measured inspiratory oxygen concentration and the estimated inspiratory carbon dioxide concentration. The expiratory volume can be calculated by rearranging equation (5) to yield:

$${V}_{e} = \frac{{V}_{i} ( 1 - {F}_{{i}_{O 2}} - {F}_{{i}_{CO 2}} )}{1 - {F}_{{e}_{O 2}} - {F}_{{e}_{CO 2}}} \qquad ( {5}^{′} )$$

Calculating the expiratory volume as a function of the measured inspiratory volume in this manner also has the effect of correlating the error in the inspiratory and expiratory volumes, which reduces the error in the estimated energy expenditure.

The processing means 7 is operable to estimated the energy expenditure of the patient 2 based on a set of measurements 6 received from the ventilator 4 and equations (1) to (5). The set of measurements 6 comprises at least one gas concentration measurement. The set of measurements preferably comprises an expiratory volume measurement (or, alternatively, an inspiratory volume measurement), an inspiratory oxygen concentration measurement and an expiratory carbon dioxide concentration measurement. The energy expenditure that is estimated by the processing means 7 is preferably also based on an expiratory oxygen concentration measurement, which may be received from an external sensor 16a or from the ventilator 4.

A new estimate of the patient's energy expenditure can be calculated using equation (3) each time that the patient breathes. To achieve this, the processing means 7 can monitor the expiratory volume, Vₑ, to detect changes indicative of the patient exhaling. Upon detecting that the patient has exhaled, new sets of measurements 6, 8 can be taken, and a new estimate of energy expenditure can be calculated using equation (3).

As mentioned above, equation (3) allows energy expenditure to be calculated in kilocalories per breath. The processing means 7 can also calculate the energy expenditure in kilocalories per day using the breathing frequency (f) measured by the ventilator 4. For a ventilator 4 that measures the breathing frequency in breaths per minute, the energy expenditure in kilocalories per day (EE′) can be calculated using the energy expenditure in kilocalories per breath (EE) and the following equation:

EE′=EE×f×60×24 (6)

The energy expenditure in kilocalories per day (EE′) is clinically more useful than the energy expenditure in kilocalories per breath (EE) because it allows the correct amount of nutrition required by the patient to be directly determined. When determining how much nutrition to give to the patient, the average (mean) value of a large number of estimates of energy expenditure in kilocalories per day is preferably used, each estimate of energy expenditure in kilocalories per day being calculated based upon a respective estimate of energy expenditure in kilocalories per breath.

The fundamental principles of estimating the energy expenditure of a patient in accordance with the present invention have thus been described. This approach can optionally be improved by performing corrections upon the set of measurements 6 received from the ventilator 4. The purpose of these corrections is to account for the fact that the ventilator 4 takes its measurements at different thermodynamic conditions from those that exist at the lungs of the patient 2. Three main factors can cause a divergence between the set of measurements 6 and the corresponding properties of the gas that is actually delivered to the lungs:
the temperature difference between the ventilator 4 and the mouth of the patient 2 as gas travels through the pulmonary tubes 10;
if there is a humidifier 22 connected between the ventilator 4 and the patient 2, the water vapour generated by the humidifier will alter the concentrations of oxygen and carbon dioxide, as well as the pressure and temperature; and
the compliance and resistance of the pulmonary tubes 10, which will alter the inspiratory volume delivered from the ventilator 4.

These factors can be corrected for using models such as: the ideal gas law; Dalton's law for adding partial pressures; and calculating the influence of compliance and resistance of the pulmonary tubes on the tidal volume.

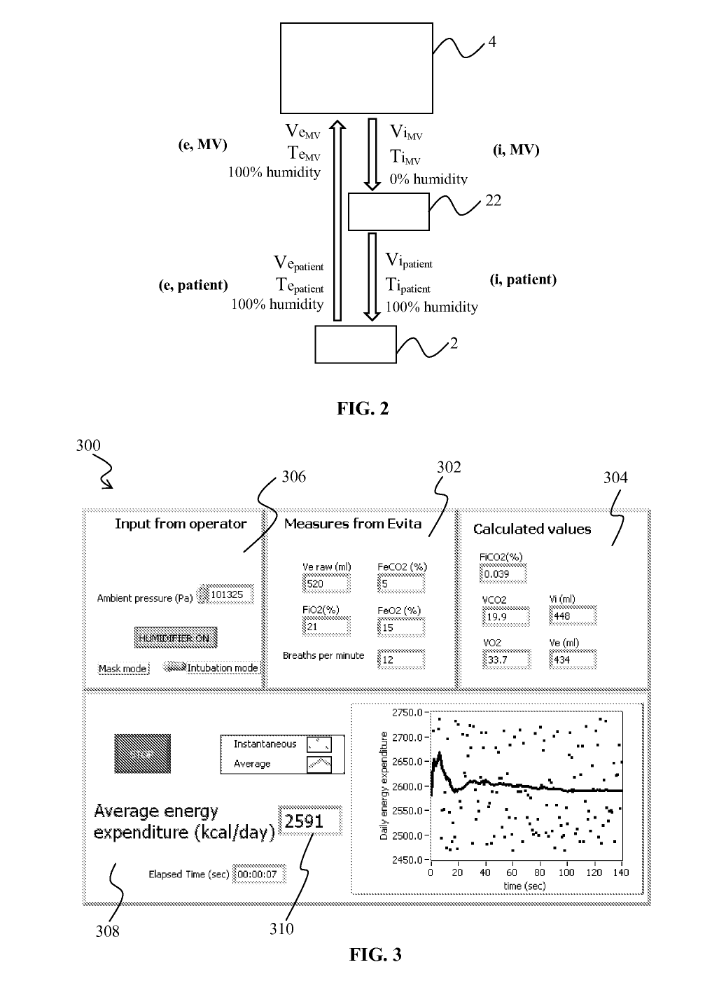

FIG. 2 illustrates the different thermodynamic conditions that exist when a humidifier 22 is connected between a ventilator 4 and a patient 2. In FIG. 2, T denotes temperature, V denotes volume, the subscript i denotes a property of the inspiratory gas, the subscript e denotes a property of the expiratory gas, the subscript patient denotes a property measured at the patient 2, and the subscript MV denotes a property measured at the ventilator 4. Thus, FIG. 2 shows that the temperatures and volumes of the inspiratory and expiratory gases differ depending upon whether they are measured at the ventilator 4 or at the patient 2. FIG. 2 also shows that the relative humidity of the inspiratory gas at the outlet of the ventilator 4 is considered to be zero, whereas the relative humidity of the inspiratory gas at the outlet of the humidifier 22 is considered to be 100%. The relative humidity of the expiratory gas is considered to be 100% at both the patient 2 and the ventilator 4.

A method of correcting the expiratory volume (Vₑ) measurement to account for the temperature difference between the ventilator 4 and the mouth of the patient 2, and also to account for the presence of a humidifier 22, will now be described.

The expiratory volume measurement that the ventilator 4 provides to the apparatus 1 as part of the set of measurements 6 is expressed in Body Temperature Pressure Saturated (BTPS) conditions. Measurements expressed in BTPS conditions assume that a gas has 100% relative humidity, a temperature of 37° C. and a pressure of 101.325 kPa. In order to express the expiratory volume measurement in BTPS conditions, the ventilator 4 automatically converts the measured expiratory volume that is actually measured to BTPS conditions, assuming that the expiratory volume was measured at a temperature of T_{y}° C. and a relative humidity of y %. For example, the Dräger Evita® XL assumes that T_{y} is 30° C. and that y is 100%. However, Equations (1), (2) and (3) assume that the expiratory volume is expressed in Normal Temperature Pressure Dry (NTPD) conditions. Measurements expressed in NTPD conditions assume that a gas has 0% relative humidity, a temperature of 20° C. and a pressure of 101.325 kPa. Thus, the processing means 7 preferably converts the expiratory volume measurement received from the ventilator 4 to NTPD conditions, taking into account that it was not measured at the assumed conditions of T_{y}° C. and y % relative humidity, but was actually measured at a temperature of Tₓ° C. and a relative humidity of x %. The processing means 7 performs this calculation using the following equation:

$${V}_{e} = {V}_{{e}_{MV}} \cdot \frac{( {T}_{y} + 273.2 ) \cdot ( 273.2 )}{310.2} \cdot \frac{P - {P}_{100 \% RH} ( 37 ° C . )}{P ( P - {P}_{100 \% RH} ( {T}_{y}^{°} C . ) )} P - {P}_{x \% RH} ( {T}_{x}^{°} C . ) \qquad ( 7 )$$

where Vₑ is the corrected expiratory volume measurement at NTPD conditions, Vₑ_{_{MV}} is the expiratory volume measurement in BTPS conditions that is provided by the ventilator 4 to the apparatus 1, P is ambient atmospheric pressure (i.e. 101.325 kPa for NTPD conditions), and P_{a % RH}(b° C.) denotes the partial pressure of water vapour at a % relative humidity and a temperature of b° C. The temperature Tₓ will depend on the temperature setting of the humidifier 22. Tₓ can be determined empirically, by measuring the temperature when calibrating the apparatus 1. For example, the sensors 16 can include a temperature sensor (not shown in FIG. 1) for measuring temperature. The temperature sensor may comprise a thermocouple. Purely by way of example, a suitable temperature sensor is a J-type exposed-junction thermocouple manufactured by National Instruments Corporation. The relative humidity x % can also be determined empirically, by measuring relative humidity when calibrating the apparatus 1. For example, the sensors 16 can include a humidity sensor (not shown in FIG. 1) for measuring humidity. Purely by way of example, a suitable humidity sensor is a HIH-4000 integrated circuit humidity sensor, manufactured by Honeywell. Alternatively, the relative humidity x % can be assumed to have a value between 90% and 100%, and preferably a value of 95%.

The corrected expiratory volume measurement at NTPD conditions (Vₑ) given by Equation (7) can be substituted into Equations (1) and (2), such that Equation (3) yields a more accurate estimate of the patient's energy expenditure.

In the alternative example described above, in which the inspiratory volume (rather than the expiratory volume) is measured by the ventilator 4, the inspiratory volume can be corrected in a similar manner. In this case, the patient's energy expenditure can be estimated based upon the corrected inspiratory volume measurement.

Whilst performing such corrections can improve the accuracy of the estimate of a patient's energy expenditure, there will always be factors affecting the estimate that cannot be identified or controlled. To demonstrate the effectiveness of the method and apparatus that is disclosed herein, the inventors have analysed the impact of systematic errors and random errors upon the energy expenditure estimate. The magnitude of systematic errors was estimated comparing each measurement in the set of measurements 6 taken by a Dräger Evita® XL ventilator with a corresponding measurement taken by an external measuring device. The magnitude of random errors was calculated from the standard deviations in repeated measurements of the same property with the ventilator. The total systematic error was found to be 8.3%, whilst the total random error was found to be 0.5%. Assuming that the systematic error and random error are uncorrelated, the total error is equal to the square root of the sum of the squares of the systematic and random errors. Thus, the total error was found to be 8.3%, i.e. (0.083²+0.005²)^{0.5}. This total error is sufficiently small for the method and apparatus that are disclosed herein to be suitable for clinical use.

FIG. 3 is an example of a user interface 300 for an apparatus for estimating the energy expenditure of a patient. The user interface 300 can be presented on the display 20 of the apparatus 1. The user interface 300 comprises a plurality of regions 302, 304, 306, 308. Region 302 is operable to display one or more measurements made by the ventilator 4 and/or the sensors 16. Region 304 is operable to display one or more values that are calculated based upon measurements made by the ventilator 4 and/or the sensors 16. Region 306 is operable to receive a user input to specify the values of parameters used to estimate the energy expenditure of a patient. For example, region 306 allows a user to specify any one or more of the following parameters: ambient atmospheric pressure; a Boolean value indicating whether a humidifier 22 is operating; and a Boolean value indicating whether the patient 2 is receiving breathing assistance via face mask or a tracheal tube. Region 308 is operable to display the energy expenditure 310 of the patient 2.

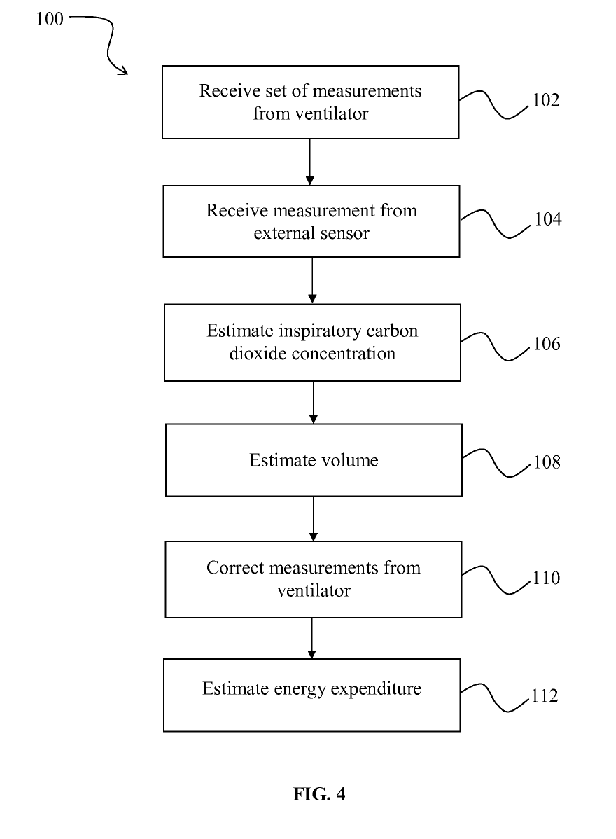

FIG. 4 is a flow diagram of a method 100 for estimating the energy expenditure of a patient 2. In step 102, a set of measurements 6 is received from the ventilator 4. As mentioned previously, the set of measurements 6 preferably comprises an expiratory volume measurement, an inspiratory oxygen concentration measurement and an expiratory carbon dioxide concentration measurement. In step 104, a measurement 8 is received from an external sensor 16. The measurement 8 that is received from the external sensor 16 is preferably an expiratory oxygen concentration measurement. Whilst FIG. 4 shows that step 104 precedes step 102, it will be appreciated that steps 102 and 104 can be performed in any order or be performed simultaneously. In step 106, the inspiratory carbon dioxide concentration is estimated. In step 108, the inspiratory volume is estimated. In step 110, one or more of the measurements in the set of measurements 6 from the ventilator 4 is corrected to produce a respective corrected measurement. The corrected measurement compensates for a difference in thermodynamic conditions existing at the ventilator 4 and the patient 2. Whilst FIG. 4 shows that steps 106 and 108 precede step 110, it will be appreciated that the corrected measurement may alternatively be performed before the inspiratory carbon dioxide concentration and inspiratory volume are estimated. In step 112, the energy expenditure of the patient 2 is estimated based upon at least the set of measurements 6 from the ventilator 4. The energy expenditure can also be based upon the expiratory oxygen concentration measurement, the estimated inspiratory carbon dioxide concentration, the estimated inspiratory volume (or estimated expiratory volume) and/or a corrected measurement.

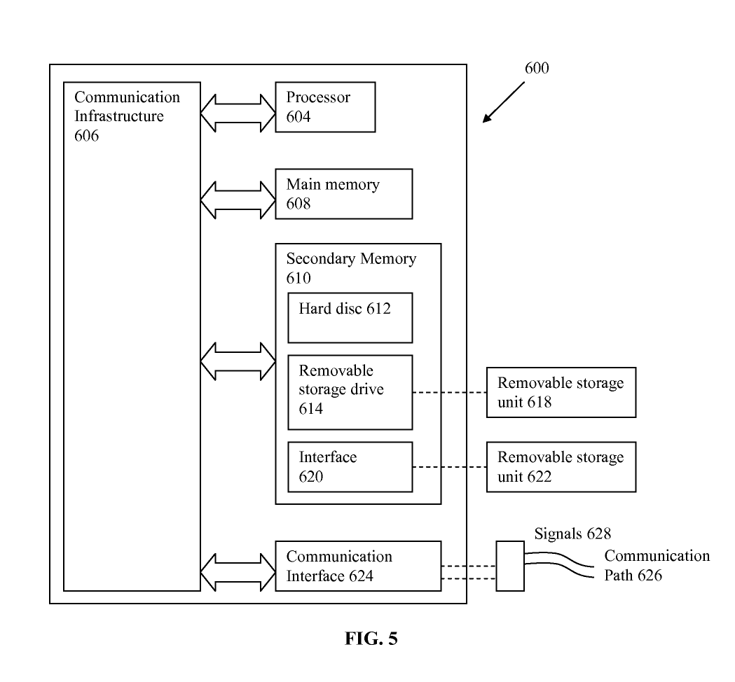

The method 100 can be implemented by a computer system 600 such as that shown in FIG. 5. The invention can also be implemented as program code for execution by the computer system 600. After reading this description, it will become apparent to a person skilled in the art how to implement the invention using other computer systems and/or computer architectures.

Computer system 600 includes one or more processors, such as processor 604. Processor 604 may be any type of processor, including but not limited to a special purpose or a general-purpose digital signal processor. Processor 604 is connected to a communication infrastructure 606 (for example, a bus or network). Computer system 600 also includes a main memory 608, preferably random access memory (RAM), and may also include a secondary memory 610. Secondary memory 610 may include, for example, a hard disk drive 612 and/or a removable storage drive 614, representing a floppy disk drive, a magnetic tape drive, an optical disk drive, etc. Removable storage drive 614 reads from and/or writes to a removable storage unit 618 in a well-known manner. Removable storage unit 618 represents a floppy disk, magnetic tape, optical disk, etc., which is read by and written to by removable storage drive 614. As will be appreciated, removable storage unit 618 includes a computer usable storage medium having stored therein computer software and/or data.

In alternative implementations, secondary memory 610 may include other similar means for allowing computer programs or other instructions to be loaded into computer system 600. Such means may include, for example, a removable storage unit 622 and an interface 620. Examples of such means may include a program cartridge and cartridge interface (such as that previously found in video game devices), a removable memory chip (such as an EPROM, or PROM, or flash memory) and associated socket, and other removable storage units 622 and interfaces 620 which allow software and data to be transferred from removable storage unit 622 to computer system 600. Alternatively, the program may be executed and/or the data accessed from the removable storage unit 622, using the processor 604 of the computer system 600.

Computer system 600 may also include a communication interface 624. Communication interface 624 allows software and data to be transferred between computer system 600 and external devices. Examples of communication interface 624 may include a modem, a network interface (such as an Ethernet card), a communication port etc. Software and data transferred via communication interface 624 are in the form of signals 628, which may be electronic, electromagnetic, optical, or other signals capable of being received by communication interface 624. These signals 628 are provided to communication interface 624 via a communication path 626. Communication path 626 carries signals 628 and may be implemented using wire or cable, fibre optics, a phone line, a wireless link, a cellular phone link, a radio frequency link, or any other suitable communication channel. For instance, communication path 626 may be implemented using a combination of channels.

The terms “computer program medium” and “computer usable medium” are used generally to refer to media such as removable storage drive 614, a hard disk installed in hard disk drive 612, and signals 628. These computer program products are means for providing software to computer system 600. However, these terms may also include signals (such as electrical, optical or electromagnetic signals) that embody the computer program disclosed herein.

Computer programs (also called computer control logic) are stored in main memory 608 and/or secondary memory 610. Computer programs may also be received via communication interface 624. Such computer programs, when executed, enable computer system 600 to implement the method described herein. Accordingly, such computer programs represent controllers of computer system 600. Where the method is implemented using software, the software may be stored in a computer program product and loaded into computer system 600 using removable storage drive 614, hard disk drive 612, or communication interface 624, to provide some examples.

In alternative embodiments, the invention can be implemented as control logic in hardware, firmware, software or any combination thereof The apparatus may be implemented by dedicated hardware, such as one or more application-specific integrated circuits (ASICs) or appropriately connected discrete logic gates. A suitable hardware description language can be used to implement the method described herein with dedicated hardware.

The method 100 can be performed by instructions stored on a processor-readable medium. The processor-readable medium may be: a read-only memory (including a PROM, EPROM or EEPROM); random access memory; a flash memory; an electrical, electromagnetic or optical signal; a magnetic, optical or magneto-optical storage medium; one or more registers of a processor; or any other type of processor-readable medium.

It will be understood that the invention has been described above purely by way of example, and that modifications of detail can be made within the scope of invention.

1. An apparatus for estimating energy expenditure of a patient, the apparatus comprising:
means for receiving a set of measurements from a ventilator, the ventilator being configured to measure at least one gas concentration measurement in order to provide breathing assistance to the patient, wherein the set of measurements comprises said at least one gas concentration measurement;
means for estimating the energy expenditure of the patient based on the set of measurements.

2. An apparatus in accordance with claim 1, wherein the at least one gas concentration measurement comprises an expiratory carbon dioxide concentration measurement.

3. An apparatus in accordance with claim 1, wherein the at least one gas concentration measurement comprises an inspiratory oxygen concentration measurement.

4. An apparatus in accordance with claim 3, wherein the apparatus further comprises means for estimating an inspiratory carbon dioxide concentration based on the inspiratory oxygen concentration measurement.

5. An apparatus in accordance with claim 1, wherein the apparatus further comprises means for estimating an inspiratory carbon dioxide concentration based on a known concentration of carbon dioxide in medical air.

6. An apparatus in accordance with claim 1, wherein the means for estimating the energy expenditure is operable to estimate the energy expenditure based on an estimate of inspiratory carbon dioxide concentration.

7. An apparatus in accordance with claim 1, wherein the apparatus further comprises means for receiving an expiratory oxygen concentration measurement, and wherein the means for estimating the energy expenditure is operable to estimate the energy expenditure based on the set of measurements and the expiratory oxygen concentration measurement.

8. An apparatus in accordance with claim 7, wherein the apparatus is operable to receive the expiratory oxygen concentration measurement from a sensor that is separate from the ventilator.

9. An apparatus in accordance with claim 1, wherein the set of measurements further comprises an expiratory volume measurement or an inspiratory volume measurement.

10. An apparatus in accordance with claim 1, wherein the apparatus further comprises means for estimating an inspiratory volume or an expiratory volume based on the set of measurements.

11. An apparatus in accordance with claim 1, wherein the means for estimating the energy expenditure is further operable to estimate the energy expenditure of the patient based on an estimate of inspiratory volume or an estimate of expiratory volume.

12. An apparatus in accordance with claim 1, wherein the apparatus further comprises means for correcting a measurement in the set of measurements to produce a corrected measurement, wherein the corrected measurement compensates for a difference between a thermodynamic condition at the ventilator and a respective thermodynamic condition at the patient, and wherein the means for estimating the energy expenditure of the patient is further operable to estimate the energy expenditure based on the corrected measurement.

13. An apparatus in accordance with claim 12, wherein the corrected measurement compensates for a difference in temperature and/or relative humidity.

14. An apparatus in accordance with claim 12, wherein the set of measurements further comprises an expiratory volume measurement, and wherein the means for correcting a measurement is operable to correct the expiratory volume measurement.

15. An apparatus in accordance with claim 1, wherein the set of measurements further comprises a respiratory frequency.

16. An apparatus in accordance with claim 1, wherein the apparatus is integrated with the ventilator.

17. An apparatus in accordance with claim 1, wherein the apparatus is detachably connectable to the ventilator.

18. A method for estimating energy expenditure of a patient, the method comprising:
receiving a set of measurements from a ventilator, the ventilator being configured to measure at least one gas concentration measurement in order to provide breathing assistance to the patient, wherein the set of measurements comprises said at least one gas concentration measurement; and
estimating the energy expenditure of the patient based on the set of measurements.

19. A method in accordance with claim 18, wherein the at least one gas concentration measurement comprises an expiratory carbon dioxide concentration measurement.

20. A method in accordance with claim 18, wherein the at least one gas concentration measurement comprises an inspiratory oxygen concentration measurement.

21. A method in accordance with claim 20, wherein the method further comprises estimating an inspiratory carbon dioxide concentration based on the inspiratory oxygen concentration measurement.

22. A method in accordance with claim 18, wherein the method further comprises estimating an inspiratory carbon dioxide concentration based on a known concentration of carbon dioxide in medical air.

23. A method in accordance with claim 18, wherein the step of estimating the energy expenditure is based on an estimate of inspiratory carbon dioxide concentration.

24. A method in accordance with claim 18, wherein the method further comprises receiving an expiratory oxygen concentration measurement, and wherein the step of estimating the energy expenditure comprises estimating the energy expenditure based on the set of measurements and the expiratory oxygen concentration measurement.

25. A method accordance with claim 24, wherein the expiratory oxygen concentration measurement is received from a sensor that is separate from the ventilator.

26. A method in accordance with claim 18, wherein the set of measurements further comprises an expiratory volume measurement or an inspiratory volume measurement.

27. A method in accordance with claim 18, wherein the method further comprises estimating an inspiratory volume or an expiratory volume based on the set of measurements.

28. A method in accordance with claim 18, wherein the step of estimating the energy expenditure is based on an estimate of inspiratory volume or an estimate of expiratory volume.

29. A method in accordance with claim 18, wherein the method further comprises correcting a measurement in the set of measurements to produce a corrected measurement, wherein the corrected measurement compensates for a difference between a thermodynamic condition at the ventilator and a respective thermodynamic condition at the patient, and wherein the step of estimating the energy expenditure of the patient further comprises estimating the energy expenditure based on the corrected measurement.

30. A method in accordance with claim 29, wherein the corrected measurement compensates for a difference in temperature and/or relative humidity.

31. A method in accordance with claim 29, wherein the set of measurements further comprises an expiratory volume measurement, and wherein the step of correcting a measurement comprises correcting the expiratory volume measurement.

32. A method in accordance with claim 18, wherein the set of measurements further comprises a respiratory frequency.

33. A processor-readable medium comprising instructions which, when executed by a processor, cause the processor to perform a method for estimating energy expenditure of a patient, the method comprising:
receiving a set of measurements from a ventilator, the ventilator being configured to measure at least one gas concentration measurement in order to provide breathing assistance to the patient, wherein the set of measurements comprises said at least one gas concentration measurement; and
estimating the energy expenditure of the patient based on the set of measurements.

34. A processor-readable medium in accordance with claim 33, wherein the at least one gas concentration measurement comprises an expiratory carbon dioxide concentration measurement.

35. A processor-readable medium in accordance with claim 33, wherein the at least one gas concentration measurement comprises an inspiratory oxygen concentration measurement.

36. A processor-readable medium in accordance with claim 35, wherein the method further comprises estimating an inspiratory carbon dioxide concentration based on the inspiratory oxygen concentration measurement.

37. A processor-readable medium in accordance with claim 33, wherein the method further comprises estimating an inspiratory carbon dioxide concentration based on a known concentration of carbon dioxide in medical air.

38. A processor-readable medium in accordance with claim 33, wherein the step of estimating the energy expenditure is based on an estimate of inspiratory carbon dioxide concentration.

39. A processor-readable medium in accordance with claim 33, wherein the method further comprises receiving an expiratory oxygen concentration measurement, and wherein the step of estimating the energy expenditure comprises estimating the energy expenditure based on the set of measurements and the expiratory oxygen concentration measurement.

40. A processor-readable medium accordance with claim 39, wherein the expiratory oxygen concentration measurement is received from a sensor that is separate from the ventilator.

41. A processor-readable medium in accordance with claim 33, wherein the set of measurements further comprises an expiratory volume measurement or an inspiratory volume measurement.

42. A processor-readable medium in accordance with claim 41, wherein the method further comprises estimating an inspiratory volume or an expiratory volume based on the set of measurements.

43. A processor-readable medium in accordance with claim 42, wherein the step of estimating the energy expenditure is based on an estimate of inspiratory volume or an estimate of expiratory volume.

44. A processor-readable medium in accordance with claim 33, wherein the method further comprises correcting a measurement in the set of measurements to produce a corrected measurement, wherein the corrected measurement compensates for a difference between a thermodynamic condition at the ventilator and a respective thermodynamic condition at the patient, and wherein the step of estimating the energy expenditure of the patient further comprises estimating the energy expenditure based on the corrected measurement.

45. A processor-readable medium in accordance with claim 44, wherein the corrected measurement compensates for a difference in temperature and/or relative humidity.

46. A processor-readable medium in accordance with claim 44, wherein the set of measurements further comprises an expiratory volume measurement, and wherein the step of correcting a measurement comprises correcting the expiratory volume measurement.

47. A processor-readable medium in accordance with claim 33, wherein the set of measurements further comprises a respiratory frequency.

48. A processor-readable medium in accordance with claim 33, wherein the processor-readable medium is integrated with the ventilator.
